(19) United States
(12) Patent Application Publication
(10) Pub. No.: US 2025/0154158 A1
(43) Pub. Date: May 15, 2025

(54) SOLID STATE FORMS OF SELTOREXANT

(71) Applicant: TEVA CZECH INDUSTRIES S.R.O., Opava-Komarov (CZ)

(72) Inventors: Pavel Kolesa, Hlubocec (CZ); Zuzana Przeczkova, Ostrava (CZ); Adela Bartova, Ostrava-Michalkovice (CZ); Jana Romanová, Hrcava (CZ)

(21) Appl. No.: 18/840,166

(22) PCT Filed: Feb. 22, 2023
(86) PCT No.: PCT/US2023/013572

§ 371 Date: Aug. 21, 2024

Related U.S. Application Data
(60) Provisional application No. 63/313,352, filed on Feb. 24, 2022.

(51) Int. Cl. C07D 487/04

(52) U.S. Cl. CPC C07D 487/04

(57) ABSTRACT

Solid state forms of Seltorexant HCl and processes for preparation thereof, and pharmaceutical compositions thereof are disclosed.

### FIELD OF THE DISCLOSURE

The present disclosure encompasses solid state forms of Seltorexant and salts thereof, in embodiments crystalline polymorphs of Seltorexant and salts thereof, processes for preparation thereof, and pharmaceutical compositions thereof.

### BACKGROUND OF THE DISCLOSURE

Seltorexant, [(3aR,6aS)-5-(4,6-Dimethyl-2-pyrimidinyl) hexahydropyrrolo[3,4-c]pyrrol-2 (1H)-yl][2-fluor-6-(2H-1,2,3-triazol-2-yl)phenyl]methanon, has the following chemical structure:

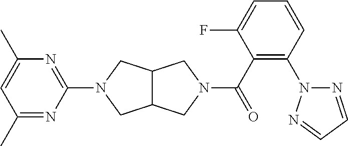 [SMILES: Cc1cc(C)nc(N2CC3CN(C(=O)c4c(F)cccc4-n4nccn4)CC3C2)n1]

Seltorexant is reported to be a selective antagonist of the human orexin-2 receptor (OX2R). Seltorexant is under clinical investigation for the treatment of major depressive disorder (MDD) with insomnia.

The compound is described in International Publication No. WO2011/050198.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms of Seltorexant.

### SUMMARY OF THE DISCLOSURE

The present disclosure provides solid state forms of Seltorexant and salts thereof, processes for preparation thereof, and pharmaceutical compositions thereof. These solid state forms can be used to prepare other solid state forms of Seltorexant, Seltorexant salts and their solid state forms.

The present disclosure also provides uses of the said solid state forms of Seltorexant and salts thereof in the preparation of other solid state forms of Seltorexant or salts thereof.

The present disclosure provides solid state forms of Seltorexant and salts thereof for use in medicine, including for the treatment of major depressive disorder (MDD) with insomnia.

The present disclosure also encompasses the use of solid state forms of Seltorexant of the present disclosure for the preparation of pharmaceutical compositions and/or formulations.

In another aspect, the present disclosure provides pharmaceutical compositions comprising solid state forms of Seltorexant according to the present disclosure.

The present disclosure includes processes for preparing the above mentioned pharmaceutical compositions. The processes include combining any one or a combination of the solid state forms of Seltorexant of the present disclosure with at least one pharmaceutically acceptable excipient.

The solid state forms of Seltorexant as defined herein and the pharmaceutical compositions or formulations of the solid state forms of Seltorexant of the present disclosure may be used as medicaments, such as for the treatment of major depressive disorder.

The present disclosure also provides methods of treating major depressive disorder, by administering a therapeutically effective amount of any one or a combination of the solid state forms of Seltorexant of the present disclosure, or at least one of the above pharmaceutical compositions, to a subject suffering from major depressive disorder (MDD) with insomnia, or otherwise in need of the treatment.

The present disclosure also provides uses of the solid state forms of Seltorexant of the present disclosure, or at least one of the above pharmaceutical compositions, for the manufacture of medicaments for treating major depressive disorder.

### BRIEF DESCRIPTION OF THE DRAWINGS

FIG. 1 shows a characteristic X-ray powder diffraction pattern (XRPD) of Seltorexant HCl amorphous form.

FIG. 2 shows a characteristic X-ray powder diffraction pattern (XRPD) of Seltorexant HCl Form C1.

FIG. 3 shows a characteristic X-ray powder diffraction pattern (XRPD) of Seltorexant HCl Form C2.

FIG. 4 shows a characteristic X-ray powder diffraction pattern (XRPD) of Seltorexant HCl Form C5.

### DETAILED DESCRIPTION OF THE DISCLOSURE

The present disclosure encompasses solid state forms of Seltorexant and salts thereof, in embodiments crystalline polymorphs of Seltorexant and salts thereof, processes for preparation thereof, and pharmaceutical compositions thereof.

Solid state properties of Seltorexant and crystalline polymorphs thereof can be influenced by controlling the conditions under which Seltorexant and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Seltorexant described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Seltorexant. In some embodiments of the disclosure, the described crystalline polymorph of Seltorexant may contain from about 1% to about 20% (w/w), from about 5% to about 20% (w/w), or from about 5% to about 10% (w/w) of one or more other crystalline polymorph of the same Seltorexant.

Depending on which other crystalline polymorphs a comparison is made, the crystalline polymorphs of Seltorexant of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Seltorexant referred to herein as being characterized by graphical data “as depicted in” or “as substantially depicted in” a Figure will thus be understood to include any crystal forms of Seltorexant characterized with the graphical data having such small variations, as are well known to the skilled person, in comparison with the Figure.

As used herein, and unless stated otherwise, the term “anhydrous” in relation to crystalline forms of Seltorexant, relates to a crystalline form of Seltorexant which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Seltorexant of the present disclosure corresponds to a crystalline polymorph of Seltorexant that is physically separated from the reaction mixture in which it is formed.

As used herein, unless stated otherwise, the XRPD measurements are taken using copper Kα radiation wavelength 1.54187 Å. XRPD peaks reported herein are measured using CuK α radiation, λ=1.54187 Å, typically at a temperature of 25±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The present disclosure includes Seltorexant Hydrochloride and solid state forms thereof.

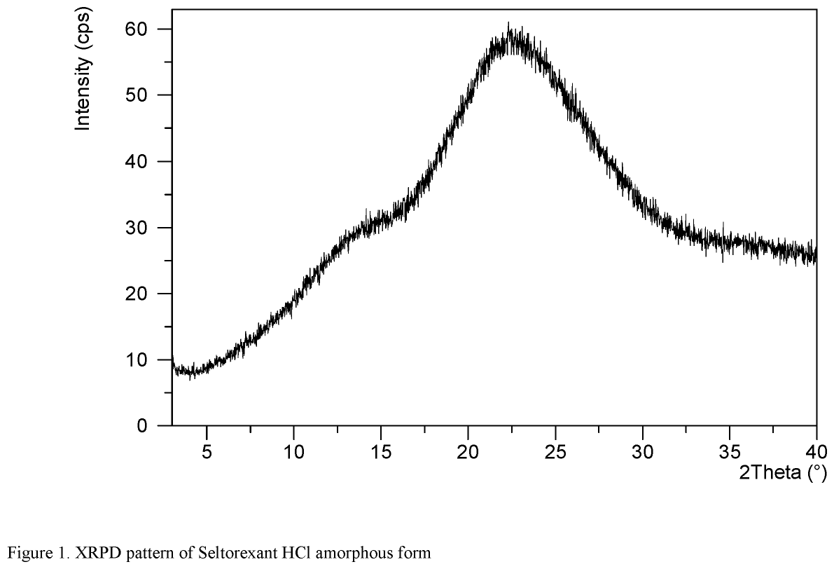

The present disclosure includes an amorphous form of Seltorexant HCl. The amorphous form of Seltorexant HCl may be characterized by an X-ray powder diffraction pattern substantially as depicted in FIG. 1.

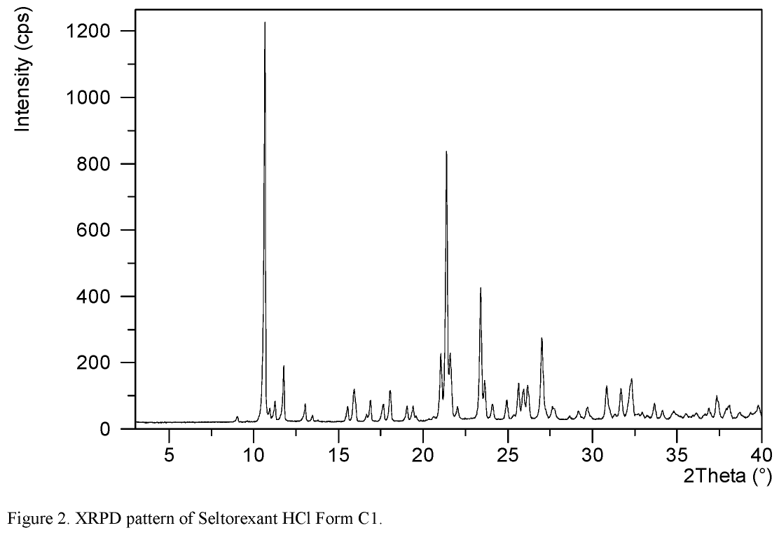

The present disclosure includes a crystalline polymorph of Seltorexant HCl, designated Form C1. The crystalline Form C1 of Seltorexant HCl may be characterized by data selected from one or more of the following: an X-ray powder diffraction pattern substantially as depicted in FIG. 2; an X-ray powder diffraction pattern having peaks at 10.7, 11.2, 11.8, 13.0 and 15.9 degrees 2-theta±0.2 degrees 2-theta; and combinations of these data.

Crystalline Form C1 of Seltorexant HCl may be further characterized by an X-ray powder diffraction pattern having peaks at 10.7, 11.2, 11.8, 13.0 and 15.9 degrees 2-theta±0.2 degrees 2-theta, and also having any one, two, three, four or five additional peaks selected from 15.5, 18.0, 19.0, 21.4 and 27.0 degrees 2-theta±0.2 degrees 2-theta.

In one embodiment of the present disclosure, crystalline Form C1 of Seltorexant HCl is isolated.

In one embodiment of the present disclosure, crystalline Form C1 of Seltorexant HCl may be a hydrate, e.g., a dihydrate.

Crystalline Form C1 of Seltorexant HCl may be characterized by an X-ray powder diffraction pattern having peaks at 10.7, 11.2, 11.8, 13.0, 15.5, 15.9, 18.0, 19.0, 21.4 and 27.0 degrees 2-theta±0.2 degrees 2-theta.

Crystalline Form C1 of Seltorexant HCl may be characterized by each of the above characteristics alone/or by all possible combinations, e.g., an XRPD pattern having peaks at 10.7, 11.2, 11.8, 13.0 and 15.9 degrees 2-theta±0.2 degrees 2-theta; an XRPD pattern as depicted in FIG. 2, and combinations thereof.

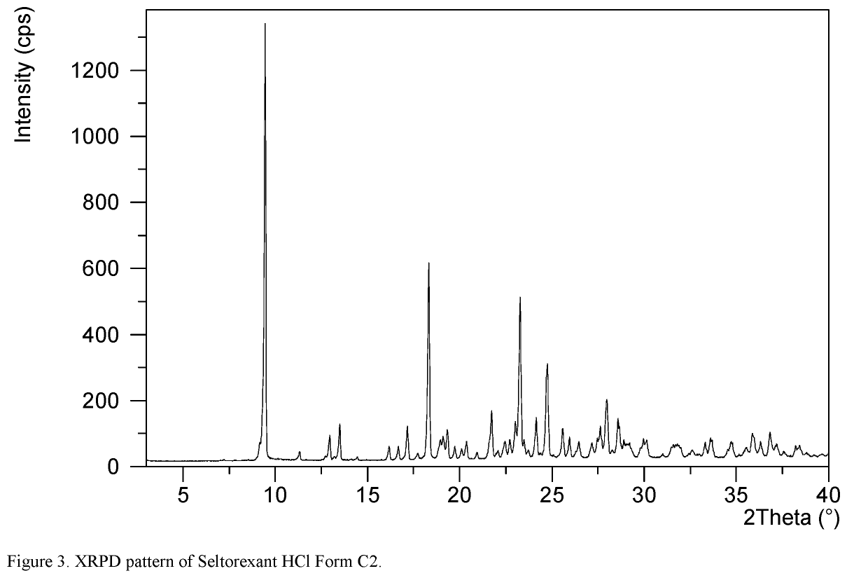

The present disclosure includes a crystalline polymorph of Seltorexant HCl, designated Form C2. The crystalline Form C2 of Seltorexant HCl may be characterized by data selected from one or more of the following: an X-ray powder diffraction pattern substantially as depicted in FIG. 3; an X-ray powder diffraction pattern having peaks at 9.5, 11.3, 13.5, 16.7 and 18.3 degrees 2-theta±0.2 degrees 2-theta; and combinations of these data.

Crystalline Form C2 of Seltorexant HCl may be further characterized by an X-ray powder diffraction pattern having peaks at 9.5, 11.3, 13.5, 16.7 and 18.3 degrees 2-theta±0.2 degrees 2-theta, and also having any one, two, three, four or five additional peaks selected from 16.2, 17.2, 21.7, 24.1 and 24.8 degrees 2-theta±0.2 degrees 2-theta.

In one embodiment of the present disclosure, crystalline Form C2 of Seltorexant HCl is isolated.

In one embodiment of the present disclosure, crystalline Form C2 of Seltorexant HCl may be a hydrate, e.g., about 3 hydrate to about 4 hydrate. In embodiment of the present disclosure, crystalline Form C2 of Seltorexant HCl is a 3.5 hydrate.

Crystalline Form C2 of Seltorexant HCl may be characterized by an X-ray powder diffraction pattern having peaks at 9.5, 11.3, 13.5, 16.2, 16.7, 17.2, 18.3, 21.7, 24.1 and 24.8 degrees 2-theta±0.2 degrees 2-theta.

Crystalline Form C2 of Seltorexant HCl may be characterized by each of the above characteristics alone/or by all possible combinations, e.g., an XRPD pattern having peaks at 9.5, 11.3, 13.5, 16.7 and 18.3 degrees 2-theta±0.2 degrees 2-theta; an XRPD pattern as depicted in FIG. 3, and combinations thereof.

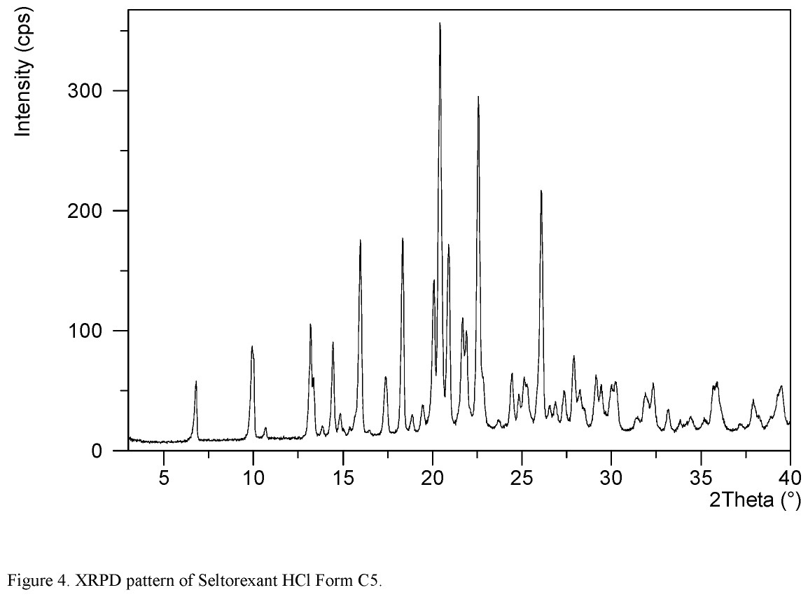

The present disclosure includes a crystalline polymorph of Seltorexant HCl, designated Form C5. The crystalline Form C5 of Seltorexant HCl may be characterized by data selected from one or more of the following: an X-ray powder diffraction pattern substantially as depicted in FIG. 4; an X-ray powder diffraction pattern having peaks at 6.8, 9.9, 14.5, 16.0 and 18.4 degrees 2-theta±0.2 degrees 2-theta; and combinations of these data.

Crystalline Form C5 of Seltorexant HCl may be further characterized by an X-ray powder diffraction pattern having peaks at 6.8, 9.9, 14.5, 16.0 and 18.4 degrees 2-theta±0.2 degrees 2-theta, and also having any one, two, three, four or five additional peaks selected from 17.4, 20.4, 20.9, 22.6 and 26.1 degrees 2-theta±0.2 degrees 2-theta.

In one embodiment of the present disclosure, crystalline Form C5 of Seltorexant HCl is isolated.

In one embodiment of the present disclosure, crystalline Form C5 of Seltorexant HCl may be anhydrous.

Crystalline Form C5 of Seltorexant HCl may be characterized by an X-ray powder diffraction pattern having peaks at 6.8, 9.9, 14.5, 16.0, 17.4, 18.4, 20.4, 20.9, 22.6 and 26.1 degrees 2-theta±0.2 degrees 2-theta.

Crystalline Form C5 of Seltorexant HCl may be characterized by each of the above characteristics alone/or by all possible combinations, e.g., an XRPD pattern having peaks at 6.8, 9.9, 14.5, 16.0 and 18.4 degrees 2-theta±0.2 degrees 2-theta; an XRPD pattern as depicted in FIG. 4, and combinations thereof.

The above crystalline forms can be used to prepare other crystalline forms of Seltorexant, Seltorexant salts and their solid state forms.

The present disclosure encompasses a process for preparing other solid state forms of Seltorexant, Seltorexant salts and their solid state forms thereof. The process includes preparing any one of the Seltorexant (salts) and solid state forms thereof as disclosed herein by the processes of the present disclosure, and converting it to other Seltorexant salt(s) and solid state forms thereof.

The present disclosure provides the above described solid state forms of Seltorexant for use in the preparation of pharmaceutical compositions comprising Seltorexant and/or solid state forms thereof. In particular the present disclosure encompasses the use of the above described solid state forms of Seltorexant and salts thereof, for the preparation of a pharmaceutical composition in the form of a tablet comprising Seltorexant or salt thereof.

The present disclosure also encompasses the use of solid state forms of Seltorexant of the present disclosure for the preparation of pharmaceutical compositions of Seltorexant and/or solid state forms thereof. In particular the present disclosure encompasses the above described solid state forms of Seltorexant and salts thereof, for the preparation of a pharmaceutical composition or formulation, preferably an oral formulation in the form of a tablet comprising Seltorexant or salt thereof.

The present disclosure includes processes for preparing the above mentioned pharmaceutical compositions. The processes include combining any one or a combination of the solid state forms of Seltorexant of the present disclosure with at least one pharmaceutically acceptable excipient.

Pharmaceutical combinations or formulations of the present disclosure contain any one or a combination of the solid state forms of Seltorexant of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., Klucel®), hydroxypropyl methyl cellulose (e.g., Methocel®), liquid glucose, magnesium aluminum silicate, maltodextrin, methylcellulose, polymethacrylates, povidone (e.g., Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Seltorexant and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Seltorexant can be administered. Seltorexant may be formulated for administration to a mammal, in embodiments to a human, by injection. Seltorexant can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Seltorexant and the pharmaceutical compositions and/or formulations of Seltorexant of the present disclosure can be used as medicaments, in embodiments in the treatment of major depressive disorder (MDD) with insomnia.

The present disclosure also provides methods of treating major depressive disorder by administering a therapeutically effective amount of any one or a combination of the crystalline polymorphs of Seltorexant of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Powder X-ray Diffraction was performed on an X-Ray powder diffractometer PanAlytical X′pert Pro; CuKα radiation (λ=1.54187 Å); X'Celerator detector with active length 2.122 degrees 2-theta; laboratory temperature 25±3° C.; zero background sample holders. Prior to analysis, the samples were gently ground using a mortar and pestle to obtain a fine powder. The ground sample was adjusted into a cavity of the sample holder and the surface of the sample was smoothed using a cover glass.

### Measurement Parameters:

Scan range: 3-40 degrees 2-theta
Scan mode: continuous
Step size: 0.0167 degrees
Step size: 42 s
Sample spin: 60 rpm
Sample holder: zero background silicon plate

### EXAMPLES

### Preparation of Starting Materials

Seltorexant can be prepared according to methods known from the literature, for example U.S. Pat. No. 8,653,263.

### Example 1: Preparation of Seltorexant HCl Form C1

Seltorexant (300 mg) was suspended in 2-butanol (1 mL) and heated to 60° C. within a period of 30 minutes. 35% hydrochloride acid (1.05 eq, 67 μl) was added at 45° C. The sample dissolved and recrystallized at 60° C. The suspension was stirred (500 rpm) at 60° C. for 1 hour. The suspension was cooled down to 0° C. within a period of 90 minutes. Suspension was filtered and dried at RT under vacuum for about 15 minutes. Seltorexant HCl Form C1 was obtained.

### Example 2: Preparation of Seltorexant HCl Amorphous Form

Seltorexant hydrochloride salt (500 mg) was dissolved in 20 mL of chloroform at 25° C. immediately. Solution was filtered. The amorphous sample was prepared by fast evaporation of the solvent on the rotary vacuum evaporator in 5 minutes.

### Example 3: Preparation of Seltorexant HCl Form C2

Seltorexant hydrochloride amorphous form (100 mg) was dissolved in 0.5 mL of water and stirred at 25° C. for 24 hours. The sample recrystallized. The suspension was filtrated and dried at RT under the vacuum for 15 minutes. Seltorexant HCl Form C2 was obtained.

### Example 4: Preparation of Seltorexant HCl Form C5

Seltorexant hydrochloride salt (40 mg) was suspended in isopropyl alcohol (0.5 mL). Sample dissolved at 25.5° C. N-heptane (1.2 mL) was added at the room temperature. The solution was stored at 5° C. The sample recrystallized after 14 days. The suspension was filtrated and dried at RT under the vacuum for 15 minutes. Seltorexant HCl Form C5 was obtained.

### Example 5: Preparation of Seltorexant HCl Form C5

Seltorexant hydrochloride salt (Form C1, 100 mg) was suspended in methyl acetate (2 mL) and heated to 70° C. within a period of about 30 minutes. The suspension was stirred at 70° C. for 1 hour. Then, the suspension was filtrated and dried at RT under the vacuum for 15 minutes. Seltorexant HCl Form C5 was obtained.

### Example 6: Preparation of Seltorexant HCl Form C5

Seltorexant hydrochloride salt (Form C1, 80 mg) was suspended in butyl acetate (1 mL) and heated to 95° C. by heating rate 0.5° C./min. The suspension was stirred at 95° C. for 30 minutes. The suspension was cooled down to 5° C. by cooling rate 1° C. . . . min-1. Then, the suspension was filtrated and dried at RT under the vacuum for 15 minutes. Seltorexant HCl Form C5 was obtained.

### Example 7: Preparation of Seltorexant HCl Form C5

Seltorexant hydrochloride salt (Form C1, 80 mg) was suspended in tetrahydrofuran (1 mL) and heated to about 62° C. by heating rate 0.5° C./min. The suspension was stirred at about 62° C. for 30 minutes. The suspension was cooled down to 5° C. by cooling rate 1° C./min. Then, the suspension was filtrated and dried at RT under the vacuum for 15 minutes. Seltorexant HCl Form C5 was obtained.

### Example 8: Preparation of Seltorexant HCl Form C5

Seltorexant hydrochloride salt (Form C1, 80 mg) was suspended in isopropyl acetate (1 mL) and heated to 85° C. by heating rate 0.5° C./min. The suspension was stirred at 85° C. for 30 minutes. The suspension was cooled down to 5° C. by cooling rate 1° C./min. Then, the suspension was filtrated and dried at RT under the vacuum for 15 minutes. Seltorexant HCl Form C5 was obtained.

### Example 9: Preparation of Seltorexant HCl Form C5

Seltorexant HCl (Form C1, 5 grams) was suspended in ethylacetate (100 mL). The suspension was heated to 70° C. over a period of 30 minutes. The suspension was stirred at 70° C. for 2 hours. The suspension was filtrated and dried under vacuum for 1 hour.

1. A crystalline form C1 Seltorexant HCl, which is characterized by data selected from one or more of the following:
i) an XRPD pattern having peaks at 10.7, 11.2, 11.8, 13.0 and 15.9 degrees 2-theta±0.2 degrees 2-theta;
ii) an XRPD pattern as depicted in FIG. 2; or
iii) a combination of i) and ii).

2. The crystalline form of Seltorexant HCl according to claim 1, which is characterized by an XRPD pattern having peaks at 10.7, 11.2, 11.8, 13.0 and 15.9 degrees 2-theta±0.2 degrees 2-theta, and also having one, two, three, four or five additional peaks selected from 15.5, 18.0, 19.0, 21.4 and 27.0 degrees 2-theta±0.2 degrees 2-theta.

3. (canceled)

4. (canceled)

5. A crystalline form C5 Seltorexant HCl, which is characterized by data selected from one or more of the following:
i) an XRPD pattern having peaks at 6.8, 9.9, 14.5, 16.0 and 18.4 degrees 2-theta±0.2 degrees 2-theta;
ii) an XRPD pattern as depicted in FIG. 4; or
iii) combination of i) and ii).

6. The crystalline form of Seltorexant HCl according to claim 5, which is characterized by an XRPD pattern having peaks at 6.8, 9.9, 14.5, 16.0 and 18.4 degrees 2-theta±0.2 degrees 2-theta, and also having any one, two, three, four or five additional peaks selected from 17.4, 20.4, 20.9, 22.6 and 26.1 degrees 2-theta±0.2 degrees 2-theta.

7. A pharmaceutical composition comprising a crystalline form according to claim 1.

8. (canceled)

9. A pharmaceutical formulation comprising a crystalline form according to claim 1, and at least one pharmaceutically acceptable excipient.

10. (canceled)

11. A pharmaceutical composition comprising a crystalline form according to claim 5.

12. A pharmaceutical formulation comprising a crystalline form according to claim 5 and at least one pharmaceutically acceptable excipient.
